(19) United States
(12) Patent Application Publication
(10) Pub. No.: US 2021/0393248 A1
(43) Pub. Date: Dec. 23, 2021

(54) METHODS FOR CARRYING OUT A CARDIAC PROCEDURE

(71) Applicant: Baylis Medical Company Inc., Montreal (CA)

(72) Inventor: Eduardo Moriyama, Richmond (CA)

(21) Appl. No.: 17/345,104

(22) Filed: Jun. 11, 2021

Related U.S. Application Data
(60) Provisional application No. 63/040,075, filed on Jun. 17, 2020.

(51) Int. Cl. A61B 17/00; A61B 18/14; A61M 29/02

(52) U.S. Cl. CPC A61B 17/00234; A61B 18/1492; A61M 29/02; A61B 2018/00601; A61B 2017/003; A61B 2017/00358; A61B 2017/00247

(57) ABSTRACT

A method for carrying out a cardiac procedure includes a. via a superior artery, advancing a perforating tip of a perforation device towards an aorta; b. positioning the perforating tip adjacent a wall of the aorta, proximate a left pulmonary artery; and c. advancing the perforating tip to perforate through the wall of the aorta and then through a wall of the left pulmonary artery, to create a pathway between the aorta and the left pulmonary artery.

### FIELD

This document relates to medical procedures. More specifically, this document relates to methods for carrying out a cardiac procedure.

### SUMMARY

The following summary is intended to introduce the reader to various aspects of the detailed description, but not to define or delimit any invention.

Methods for carrying out a cardiac procedure are disclosed. According to some aspects, a method for carrying out a cardiac procedure includes: a. via a superior artery, advancing a perforating tip of a perforation device towards an aorta; b. positioning the perforating tip adjacent a wall of the aorta, proximate a left pulmonary artery; and c. advancing the perforating tip to perforate through the wall of the aorta and then through a wall of the left pulmonary artery, to create a pathway between the aorta and the left pulmonary artery.

In some examples, the superior artery is a right common carotid artery.

In some examples, the method further includes: d. advancing a snare towards the left pulmonary artery; and e. after step c., snaring the perforation device with the snare. Step d. can include advancing the snare towards the left pulmonary artery via a femoral vein, a hepatic vein, or a superior vein.

In some examples, the method further includes: d. via the superior artery, advancing a dilator over the perforation device; and e. after step c., advancing a dilating tip of the dilator over the perforation device and through the pathway to dilate the pathway. The dilator can be a steerable dilator.

In some examples, the method further includes: f. via the superior artery, advancing a sheath over the perforation device and over the dilator. The sheath can be a steerable sheath.

In some examples, the perforation device is a radiofrequency perforation device, the perforating tip includes a radiofrequency electrode, and step c. includes delivering radiofrequency energy to the radiofrequency electrode.

In some examples, the perforation device is a mechanical perforation device.

In some examples, the method further includes: d. delivering a therapeutic device to the pathway. The therapeutic device can be delivered over the perforation device. Step d. can include positioning a shunt in the pathway or positioning a stent in the pathway.

In some examples, at least one of fluoroscopy, angiography, electro-anatomical mapping, intracardiac echocardiography, and transesophageal echocardiography is carried out concurrently with at least one of steps a. to c.

In some examples, the method further includes confirming the creation of the pathway with at least one of fluoroscopy, electro-anatomical mapping, pressure measurement, contrast injection, and echocardiography.

In some examples, the method further includes advancing a balloon catheter over the perforation device to dilate the pathway.

In some examples, the method further includes using an anchor system to bring the aorta and the left pulmonary artery together.

In some examples, the method further includes: after step c., advancing the perforating tip out of the body towards a venous access site. The method can further include delivering a therapeutic device over the perforation device towards the pathway, via the venous access site.

### BRIEF DESCRIPTION OF THE DRAWINGS

The accompanying drawings are for illustrating examples of articles, methods, and apparatuses of the present disclosure and are not intended to be limiting. In the drawings:

FIG. 1 is a perspective view of an example perforation system;

FIG. 2 is a schematic view showing a step of a method for carrying out a cardiac procedure;

FIG. 3 is a schematic view showing a step subsequent to that of FIG. 2;

FIG. 4 is a schematic view showing a step subsequent to that of FIG. 3;

FIG. 5 is a schematic view showing a step subsequent to that of FIG. 4;

FIG. 6 is a schematic view showing a step subsequent to that of FIG. 5;

FIG. 7 is a schematic view showing a step subsequent to that of FIG. 6;

FIG. 8 is a schematic view showing a step subsequent to that of FIG. 7;

FIG. 9 is a schematic view showing a step subsequent to that of FIG. 8;

FIG. 10 is a schematic view showing a step subsequent to that of FIG. 9;

FIG. 11 is a schematic view showing a step subsequent to that of FIG. 10;

FIG. 12 is a schematic view showing a step subsequent to that of FIG. 13;

FIG. 13 is a schematic view showing a step subsequent to that of FIG. 12; and

FIG. 14 is a schematic view showing a step subsequent to that of FIG. 13.

### DETAILED DESCRIPTION

Various apparatuses or processes or compositions will be described below to provide an example of an embodiment of the claimed subject matter. No example described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Generally disclosed herein are methods for carrying out cardiac procedures, and more specifically, cardiac procedures in which a pathway (also referred to as a “communication”) is created between the aorta (e.g. the descending aorta) and the left pulmonary artery of a patient. Such procedures can be carried out, for example, to allow for the insertion of a therapeutic device (e.g. a shunt or a stent) into the pathway, to treat idiopathic pulmonary arterial hypertension or other heart defects.

The methods disclosed herein involve the creation of a pathway between the aorta and the left pulmonary artery via a superior approach—that is, the aorta can be approached via a superior artery (e.g. the right common carotid artery), and a perforation can be created in the wall of the aorta and then into the wall of the left pulmonary artery. A superior approach can allow for direct access and a less tortuous path to the aorta, length management of the devices used in the procedure, and an ideal force-vector for dilation and placement of an end-therapy device.

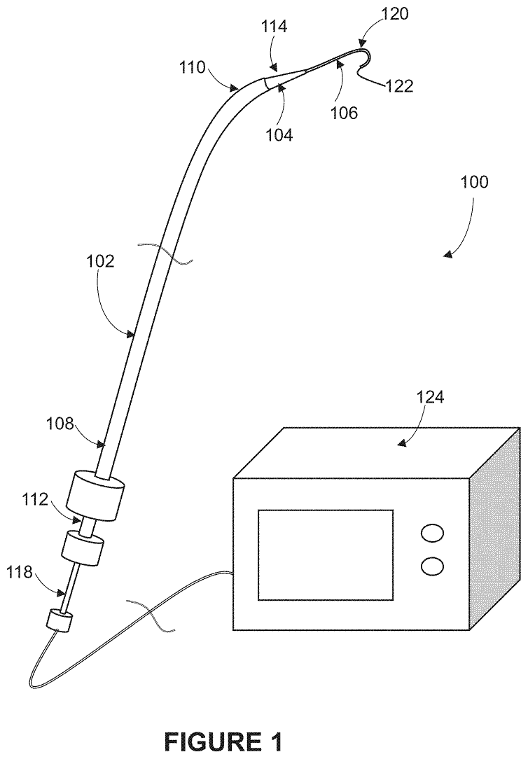

Referring first to FIG. 1, a system 100 for carrying out a cardiac procedure is shown. In the example shown, the system 100 is a perforation system, and includes a sheath 102, a dilator 104, and a perforation device 106.

The sheath 102 can be used to guide various other devices (e.g. the dilator 104, or therapeutic devices such as a stent or shunt) towards a target location in a patient's body (e.g. the aorta). The sheath 102 has a proximal portion 108 and a distal portion 110, and a lumen (not shown) extends through the sheath 102 from the proximal portion to the distal portion 110. The sheath 102 can optionally have a fixed curve, or can be steerable (i.e. the curve can be changed). A larger steerable dilator (not shown) may also replace the sheath 102 and dilator 104.

The dilator 104 can be used to dilate a perforation, and has a proximal portion 112 and a distal portion 114 having a dilating tip. The dilator 104 can optionally have a fixed curve, or can be steerable (i.e. the curve can be changed).

The perforation device 106 can be used to perforate a target anatomical structure (e.g. a wall of the aorta), and has a proximal portion 118 and a distal portion 120. The distal portion 120 has a perforating tip 122, and in the example shown is biased towards a J-shape, to prevent inadvertent perforation of anatomical structures with the perforating tip 122. In the example shown, the perforation device 106 is a radiofrequency (RF) perforation device, and the perforating tip 122 includes a radiofrequency perforation electrode. The system 100 further includes a radiofrequency generator 124, which can be connected to the perforation device 106 to deliver RF energy to the perforation electrode 122, and to one or more grounding pads (not shown).

In alternative examples, the perforation device can be a mechanical perforation device, and the perforating tip can include a sharp point.

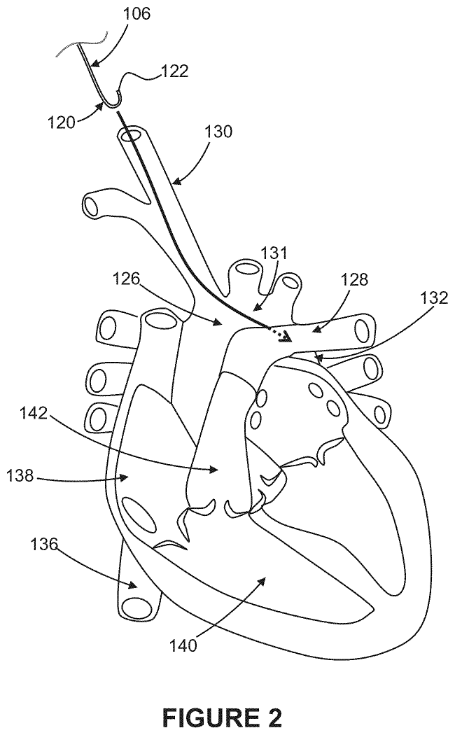

Referring now to FIGS. 2 to 14, a method for carrying out a cardiac procedure, and specifically for creating a pathway between an aorta 126 and a left pulmonary artery 128, will be described. The method will be described with regard to the system 100 shown in FIG. 1; however, the method is not limited to the system 100.

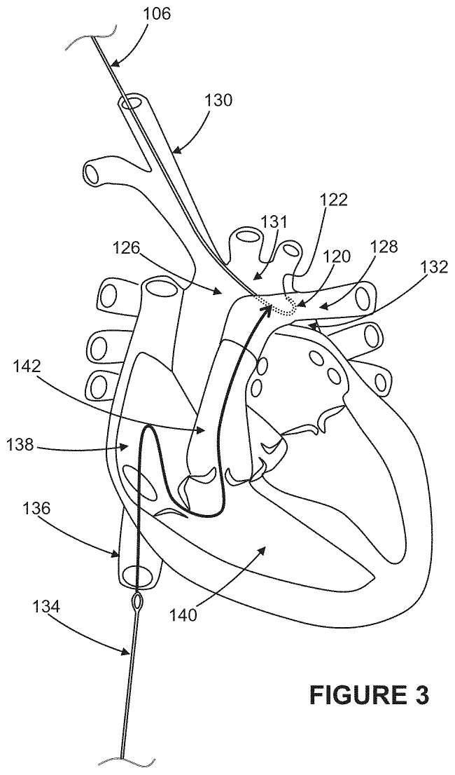

As a first step (not shown), a superior artery can be percutaneously accessed, for example using a procedure such as a Seldinger technique. Then, as shown in FIG. 2, the perforating tip 122 of the perforation device 106 can be advanced into the superior artery 130 and towards the aorta 126. In the example shown, the superior artery 130 is the common carotid artery, and the perforating tip 122 is advanced via the common carotid artery towards the aortic arch 131 and then into the descending aorta 132, as shown in FIG. 3. In FIGS. 2 to 4, features (e.g. the perforating tip 122) that are within the descending aorta 132 are shown in dotted line, as much of the descending aorta 132 is behind the left pulmonary artery 128.

Optionally, during advancement of the perforating tip 122, the position of the perforating tip 122 can be confirmed using fluoroscopy (e.g. in examples wherein the perforation device 106 includes one or more radiopaque markers or features), angiography, electro-anatomical mapping (EAM) (e.g. to confirm real-time positioning of the perforating tip 122 using real-time or pre-determined computerized tomography data, in conjunction with a catheter or guidewire with one or more EAM markers in the right atrium 138), intracardiac and/or transesophageal echocardiography (ICE and/or TEE) (e.g. using echogenic markers or features on the perforation device 106).

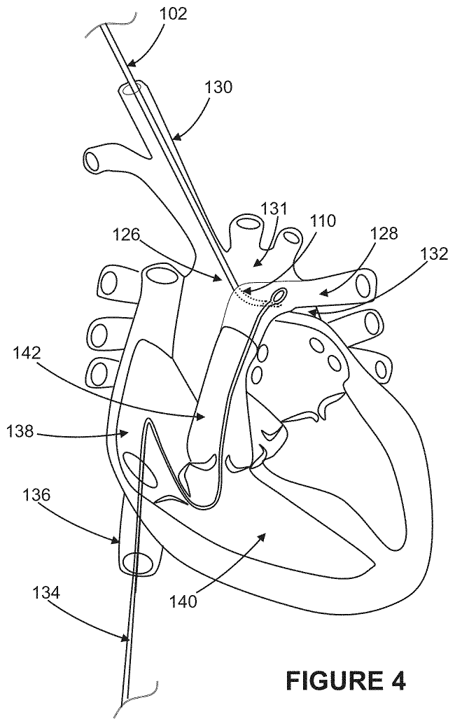

Referring still to FIG. 3, as a next step (or earlier or later in the method, for example prior to the previous steps or after the step shown in FIG. 6), a snare 134 can be advanced percutaneously towards the left pulmonary artery 128. The snare 134 can be advanced, for example, via a venous access site (not shown) such as the femoral vein, the hepatic vein, or a superior vein. In the example shown, the snare 134 is advanced via the femoral vein, the inferior vena cava 136, the right atrium 138, the right ventricle 140, and the pulmonary trunk 142, until the snare 134 reaches the left pulmonary artery 128, as shown in FIG. 4.

Referring still to FIG. 4, as a next step, the dilator 104 (not clearly visible and not labelled in FIG. 4) and the sheath 102 may be advanced towards the descending aorta 132 via the superior artery 130 and the aortic arch 131. The dilator 104 and sheath 102 can be advanced together over the perforation device 106, with the dilator 104 received in the sheath 102, or can be advanced in sequence, e.g. by advancing the dilator 104 over the perforation device 106 and then advancing the sheath 102 over the dilator 104. The dilator 104 and sheath 102 can be advanced until the dilating tip of the dilator 104 is flush with the perforating tip 122 of the perforation device (not visible in FIG. 4).

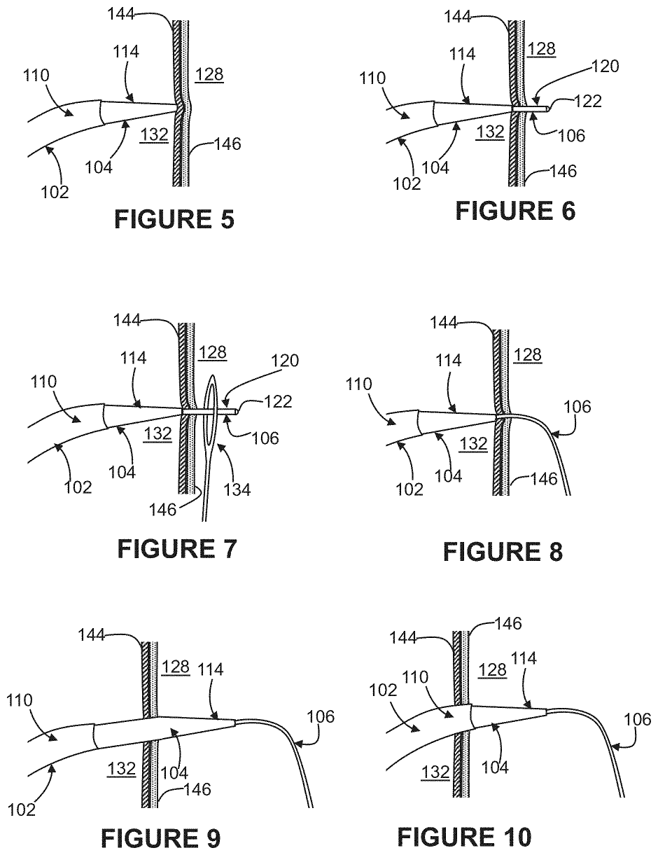

With the distal portion 110 of the sheath 102, distal portion 114 of the dilator 104, and distal portion 120 of the perforation device 106 in the descending aorta 132, the sheath 102 and dilator 104 can be maneuvered to direct the perforating tip 122 towards a desired perforation site—i.e. the wall 144 of the descending aorta 132, proximate a wall 146 of the left pulmonary artery 128—as shown in FIG. 5. This can be achieved by steering the sheath 102 and/or dilator 104 (in examples wherein the sheath 102 and/or the dilator 102 are steerable), or by adjusting the position of the sheath 102 and/or dilator 104. This step can optionally be facilitated using fluoroscopy (e.g. in examples wherein the perforation device 106 includes one or more radiopaque markers or features), angiography, electro-anatomical mapping (EAM) (e.g. to confirm real-time positioning of the perforating tip 122 using real-time or pre-determined computerized tomography data, in conjunction with a catheter or guidewire with one or more EAM markers in the right atrium 138), intracardiac and/or transesophageal echocardiography (ICE and/or TEE) (e.g. using echogenic markers or features on the perforation device 106).

Referring to FIG. 6, the perforating tip 122 can then be advanced to perforate through the wall 144 of the descending aorta 132 and through the wall 146 of the left pulmonary artery 128, to create a pathway between the descending aorta 132 and the left pulmonary artery 128. More specifically, the radiofrequency electrode can be energized with radiofrequency energy, and the perforation device 106 can be advanced to perforate through the wall 144 of the descending aorta 132 and through the wall 146 of the left pulmonary artery 128. Optionally, creation of the pathway can be confirmed using fluoroscopy, electro-anatomical mapping, pressure measurement, contrast injection, and intracardiac and/or transesophageal echocardiography.

Referring to FIGS. 7 and 8, the snare 134 can then be used to capture the distal portion 120 of the perforation device 106, and the snare 134 can then be retracted to advance the perforation device 106 towards the venous access site, optionally to externalize the perforating tip 122. Optionally, the perforation device 106 can be “flossed” to enlarge the pathway.

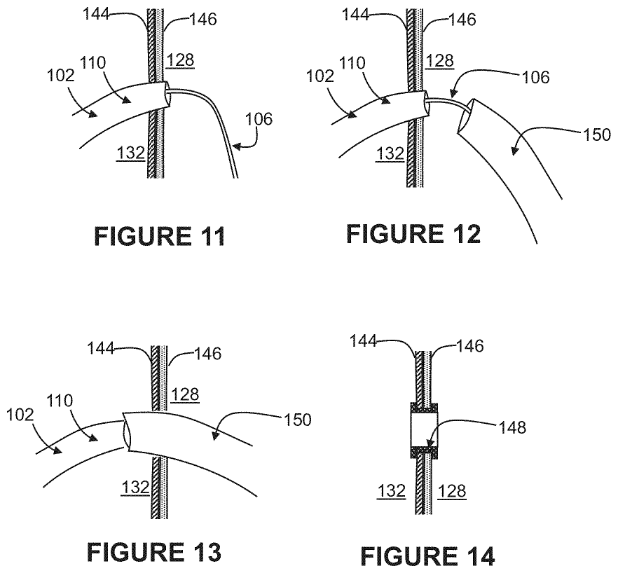

Referring to FIG. 9, the dilating tip of the dilator 104 can then be advanced over the perforation device 106 and through the pathway, to dilate the pathway. Referring to FIG. 10, the sheath 102 can then be advanced over the dilator 104 and through the pathway. Referring to FIG. 11, the dilator 104 (not visible in FIG. 11) can then be retracted back towards the superior artery 130 (not visible in FIG. 11) and removed from the body, leaving the sheath 102 and the perforation device in place 106 in the pathway.

Optionally, after perforation, the perforation device 106 can be exchanged for another wire (either via the arterial access site or the venous access site), such as a relatively stiff guidewire.

Optionally, after perforation, an anchor device such as a balloon can be advanced via the sheath 102 and used to bring the descending aorta 132 and the left pulmonary artery 128 together.

Optionally, after perforation, a balloon catheter can be advanced over the perforation device 106 and via the sheath 102 to enlarge the pathway.

With the sheath 102 and the perforation device 106 (or another guidewire) in place in the pathway, a therapeutic device can be delivered to the pathway, over the perforation device 106. The therapeutic device can be for, example, a shunt (e.g. shunt 148, shown in FIG. 14) or a stent that is positioned in the pathway. Delivery of the therapeutic device can optionally be achieved by delivering the therapeutic device over the perforation device 106 via the venous access site. For example, referring to FIG. 12, a secondary sheath 150 (e.g. a large bore sheath) can be advanced over the perforation device 106 via the venous access site, to the left pulmonary artery 128. The secondary sheath 150 can be advanced into the left pulmonary artery 128 to contact the distal portion 110 of the sheath 102. The sheath 102 can then be retracted from the pathway while advancing the secondary sheath 150 through the pathway, as shown in FIG. 13. The secondary sheath 150 can then be used to deliver the therapeutic device, i.e. shunt 148, as shown in FIG. 14. Alternatively, the therapeutic device can be delivered over the perforation device 106 via the superior artery 130 and through the sheath 102.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

We claim:

1. A method for carrying out a cardiac procedure, comprising:
a. via a superior artery, advancing a perforating tip of a perforation device towards an aorta;
b. positioning the perforating tip adjacent a wall of the aorta, proximate a left pulmonary artery; and
c. advancing the perforating tip to perforate through the wall of the aorta and then through a wall of the left pulmonary artery, to create a pathway between the aorta and the left pulmonary artery.

2. The method of claim 1, wherein the superior artery is a right common carotid artery.

3. The method of claim 1, further comprising:
d. advancing a snare towards the left pulmonary artery; and
e. after step c., snaring the perforation device with the snare.

4. The method of claim 3, wherein step d. comprises advancing the snare towards the left pulmonary artery via a femoral vein, a hepatic vein, or a superior vein.

5. The method of claim 1, further comprising:
d. via the superior artery, advancing a dilator over the perforation device; and
e. after step c., advancing a dilating tip of the dilator over the perforation device and through the pathway to dilate the pathway.

6. The method of claim 5, wherein the dilator is a steerable dilator.

7. The method of claim 5, further comprising:
f. via the superior artery, advancing a sheath over the perforation device and over the dilator.

8. The method of claim 7, wherein the sheath is a steerable sheath.

9. The method of claim 1, wherein the perforation device is a radiofrequency perforation device, the perforating tip comprises a radiofrequency electrode, and step c. comprises delivering radiofrequency energy to the radiofrequency electrode.

10. The method of claim 1, wherein the perforation device is a mechanical perforation device.

11. The method of claim 1, further comprising: d. delivering a therapeutic device to the pathway.

12. The method of claim 11, wherein the therapeutic device is delivered over the perforation device.

13. The method of claim 11, wherein step d. comprises positioning a shunt in the pathway.

14. The method of claim 11, wherein step d. comprises positioning a stent in the pathway.

15. The method of claim 1, wherein at least one of fluoroscopy, angiography, electro-anatomical mapping, intracardiac echocardiography, and transesophageal echocardiography is carried out concurrently with at least one of steps a. to c.

16. The method of claim 1, further comprising confirming the creation of the pathway with at least one of fluoroscopy, electro-anatomical mapping, pressure measurement, contrast injection, and echocardiography.

17. The method of claim 1, further comprising advancing a balloon catheter over the perforation device to dilate the pathway.

18. The method of claim 1, further comprising using an anchor system to bring the aorta and the left pulmonary artery together.

19. The method of claim 1, further comprising, after step c., advancing the perforating tip out of the body towards a venous access site.

20. The method of claim 19, further comprising delivering a therapeutic device over the perforation device towards the pathway, via the venous access site.
